(19) United States
(12) Patent Application Publication
(10) Pub. No.: US 2007/0268994 A1
(43) Pub. Date: Nov. 22, 2007

(54) X- Ray System For Use in Image Guided Procedures

(76) Inventor: Guang-Hong Chen, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/743,184

(22) Filed: May 2, 2007

Related U.S. Application Data
(60) Provisional application No. 60/796,655, filed on May 2, 2006.

(51) Int. Cl. H05G 1/60; A61B 6/00; G01N 23/00; G21K 1/12

(52) U.S. Cl. 378/4

(57) ABSTRACT

An x-ray system for use with image-guided medical procedures is programmed to move in any of a plurality of stored scan paths to acquire cone beam attenuation data from which a three-dimensional image is reconstructed. The x-ray system is programmed to move in any of the plurality of different scan paths that enable sufficient cone-beam data to be acquired.

### CROSS REFERENCE TO RELATED APPLICATION

This application claims the benefit of U.S. Provisional Application Ser. No. 60/796,655 filed on May 2, 2006 and entitled “Scan Trajectories for Large FOV Cone-Beam CT.”

### STATEMENT REGARDING FEDERALLY SPONSORED RESEARCH

This invention was made with government support under Grant No. NIH CA109992 awarded by the National Institute of Health. The United States Government has certain rights in this invention.

### BACKGROUND OF THE INVENTION

The field of the invention is medical imaging and particularly the acquisition of x-ray images for use in image-guided medical procedures.

X-ray systems for use during image-guided medical procedures include a table which is fully accessible to attending physicians for supporting the patient being treated. A gantry having a C-shaped arm supports the x-ray source at one end and the x-ray detector at its other end. The C-arm can be manipulated such that the x-ray source and detector are positioned on opposite sides of the patient, and they are spaced apart sufficiently to allow the C-arm to move them to different orientations without engaging the patient or the supporting table. As a result, the field of view (FOV) of the acquired images is large by comparison with a typical CT system.

Cone-Beam CT systems have been introduced to help guide the neuro-interventions (such as stroke interventions) and radiation therapy. The on-board cone-beam CT imaging system such as that described in U.S. Pat. No. 6,888,919 provides clinicians unique, three-dimensional anatomical information and physiological information. However, the current flat-panel based cone-beam CT systems only acquire data over a single arc/circle scanning trajectory. The single arc/circle scanning path produced by movement of the C-arm does not generate enough cone-beam projection data to reconstruct an artifact free three-dimensional image for a large image volume.

### SUMMARY OF THE INVENTION

The present invention is an x-ray imaging system that may be used for image-guided medical procedures which includes: a cone-beam x-ray source; a two-dimensional x-ray detector array; a drive mechanism for moving the x-ray source and x-ray detector about a subject positioned therebetween in a programmed path; a stored program for moving the x-ray source and x-ray detector along a scan path and acquiring a data set from which a 3D image is reconstructed. A number of stored scan paths are available that acquire sufficient cone-beam data to reconstruct a 3D image.

This invention exploits the motion capability of a c-arm gantry and robotic motion capability of a radiation therapy unit. New cone-beam CT scanning paths are employed to eliminate cone-beam artifacts for a large imaging volume which is a key requirement for lung cancer imaging, neuro-imaging of a human head, and for abdominal imaging. These novel scanning paths include a circle/arc plus one or multiple straight line(s) scan, a circle/arc plus one or multiple arc(s) scan, and the synchronized motion of gantry and patient bed to generate one or multiple twisted helical scan.

### BRIEF DESCRIPTION OF THE DRAWINGS

FIGS. 1A and 1B are perspective views of an x-ray system which employs a preferred embodiment of the present invention;

FIG. 2 is a schematic block diagram of the x-ray system of FIG. 1;

FIG. 3 is a pictorial view of an x-ray source and detector array which forms part of the x-ray system of FIG. 1;

FIGS. 4-9 are pictorial representations of scan paths performed by the x-ray system of FIG. 1 to acquire x-ray attenuation data; and

FIG. 10 is a top view of the scan path in FIG. 6.

### DETAILED DESCRIPTION OF THE PREFERRED EMBODIMENT

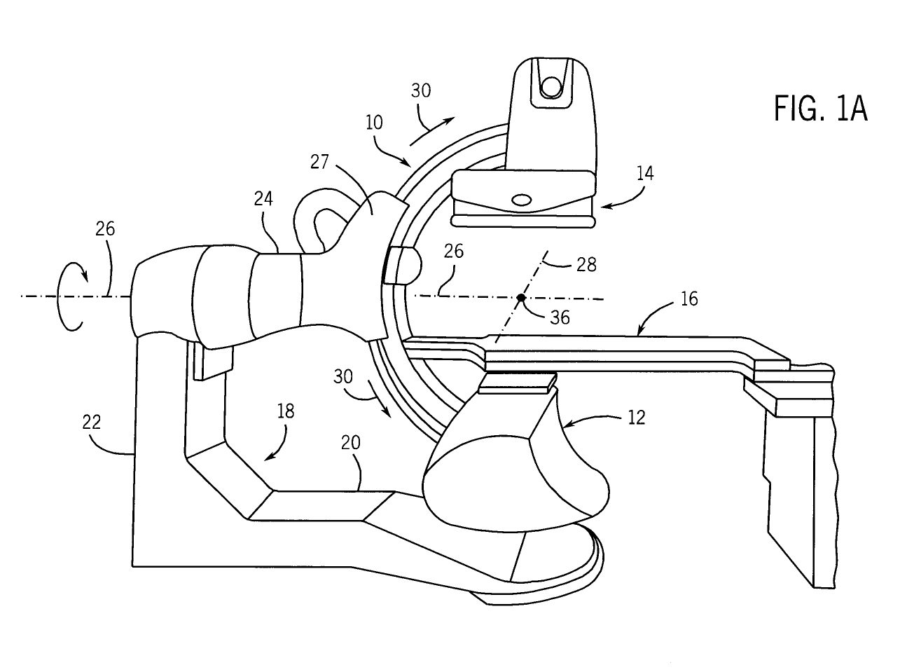

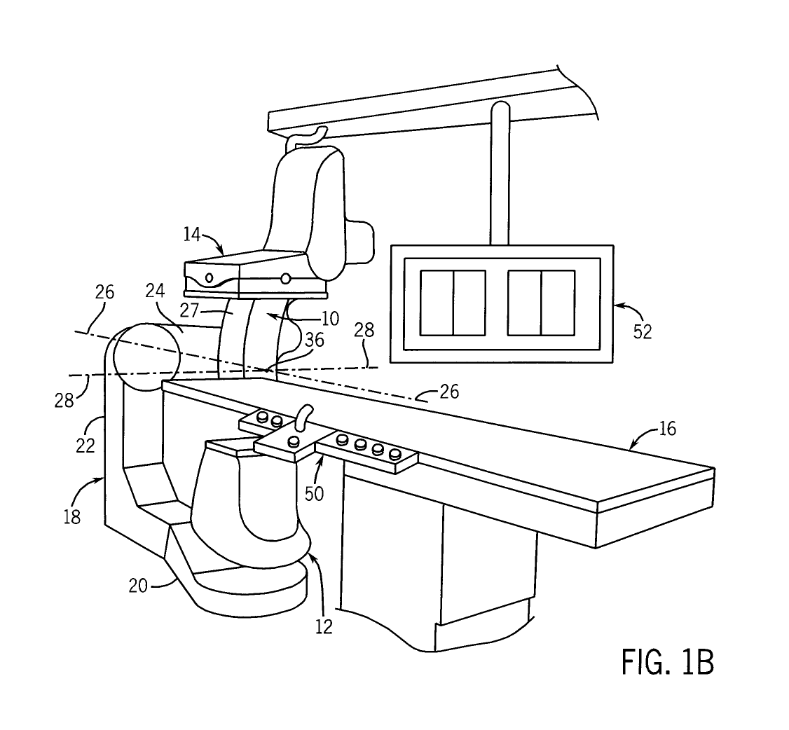

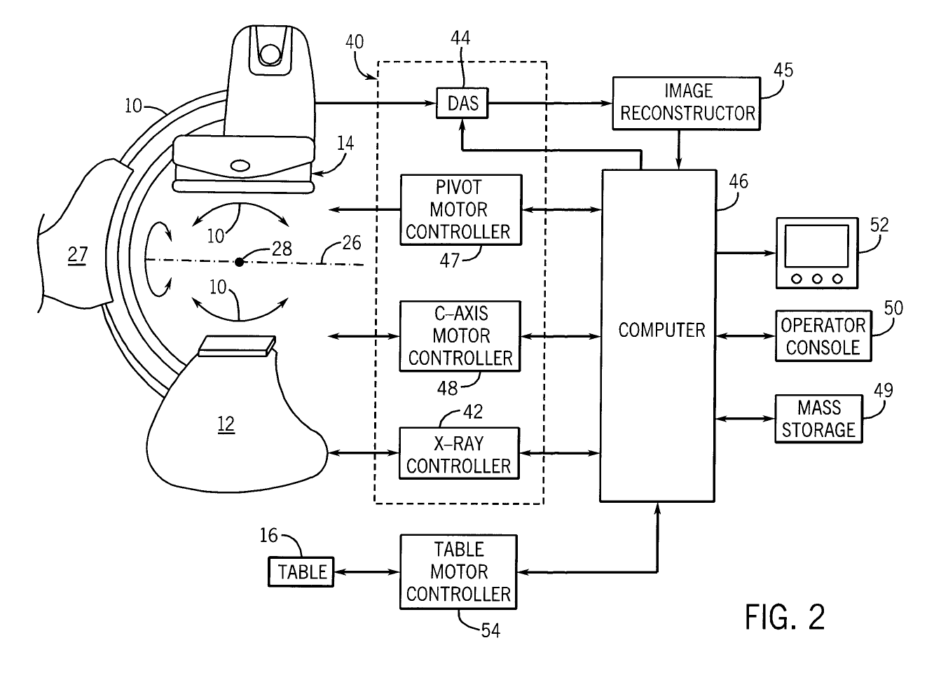

Referring particularly to FIGS. 1 and 2, the preferred embodiment of the invention employs an x-ray system that is designed specifically for use in connection with interventional procedures. It is characterized by a gantry having a C-arm 10 which carries an x-ray source assembly 12 on one of its ends and an x-ray detector array assembly 14 at its other end. The gantry enables the x-ray source 12 and detector 14 to be oriented in different positions and angles around a patient disposed on a table 16, while enabling a physician access to the patient.

The gantry includes an L-shaped pedestal 18 which has a horizontal leg 20 that extends beneath the table 16 and a vertical leg 22 that extends upward at the end of the horizontal leg 20 that is spaced from of the table 16. A support arm 24 is rotatably fastened to the upper end of vertical leg 22 for rotation about a horizontal pivot axis 26. The pivot axis 26 is aligned with the centerline of the table 16 and the arm 24 extends radially outward from the pivot axis 26 to support a C-arm drive assembly 27 on its outer end. The C-arm 10 is slidably fastened to the drive assembly 27 and is coupled to a drive motor (not shown) which slides the C-arm 10 to revolve it about a C-axis 28 as indicated by arrows 30. The pivot axis 26 and C-axis 28 intersect each other at an isocenter 36 located above the table 16 and they are perpendicular to each other.

The x-ray source assembly 12 is mounted to one end of the C-arm 10 and the detector array assembly 14 is mounted to its other end. As will be discussed in more detail below, the x-ray source 12 emits a cone beam of x-rays which are directed at the detector array 14. Both assemblies 12 and 14 extend radially inward to the pivot axis 26 such that the center ray of this cone beam passes through the system isocenter 36. The center ray of the cone beam can thus be rotated about the system isocenter around either the pivot axis 26 or the C-axis 28, or both during the acquisition of x-ray attenuation data from a subject placed on the table 16.

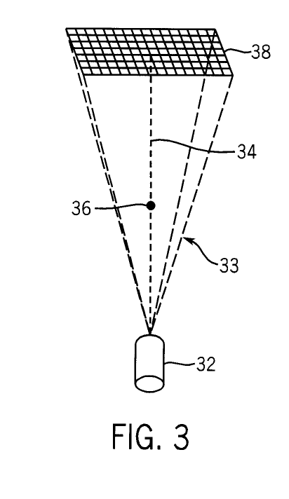

As shown in FIG. 3, the x-ray source assembly 12 contains an x-ray source 32 which emits a cone beam 33 of x-rays when energized. The center ray 34 passes through the system isocenter 36 and impinges on a two-dimensional flat panel digital detector 38 housed in the detector assembly 14. The detector 38 is a 2048 by 2048 element two-dimensional array of detector elements having a size of 41 cm by 41 cm. Each element produces an electrical signal that represents the intensity of an impinging x-ray and hence an indication of the attenuation of the x-ray as it passes through the patient. During a scan the x-ray source 32 and detector array 38 are rotated about the system isocenter 36 to acquire x-ray attenuation projection data from different angles. The detector array is able to acquire 30 projections, or views, per second and this is the limiting factor that determines how many views can be acquired for a prescribed scan path and speed.

Referring particularly to FIG. 2, the rotation of the assemblies 12 and 14 and the operation of the x-ray source 32 are governed by a control mechanism 40 of the CT system. The control mechanism 40 includes an x-ray controller 42 that provides power and timing signals to the x-ray source 32. A data acquisition system (DAS) 44 in the control mechanism 40 samples data from detector elements 38 and passes the data to an image reconstructor 45. The image reconstructor 45, receives digitized x-ray data from the DAS 44 and performs high speed image reconstruction. The reconstructed image is applied as an input to a computer 46 which stores the image in a mass storage device 49 or processes the image further to produce parametric images according to the teachings of the present invention.

The control mechanism 40 also includes pivot motor controller 47 and a C-axis motor controller 48. In response to motion commands from the computer 46 the motor controllers 47 and 48 provide power to motors in the x-ray system that produce the rotations about respective pivot axis 26 and C-axis 28. As will be discussed below, a program executed by the computer 46 generates motion commands to the motor drives 47 and 48 to move the assemblies 12 and 14 in a prescribed scan path.

The computer 46 also receives commands and scanning parameters from an operator via console 50 that has a keyboard and other manually operable controls. An associated cathode ray tube display 52 allows the operator to observe the reconstructed image and other data from the computer 46. The operator supplied commands are used by the computer 46 under the direction of stored programs to provide control signals and information to the DAS 44, the x-ray controller 42 and the motor controllers 47 and 48. In addition, computer 46 operates a table motor controller 54 which controls the motorized table 16 to position the patient with respect to the system isocenter 36. The computer 46 stores programs which enable it to perform very different scans. These will be described in more detail below.

There are three difficulties commonly encountered when reconstructing 3D images from cone beam data sets. First, artifacts will be produced in the 3D image if the cone-beam projection data is not acquired from an appropriate design of the x-ray source orbit. This is a geometric problem of not acquiring views from a sufficient number of view angles and is common to cone beam acquisitions with conventional CT systems that employ a single circular acquisition path. This data sufficiency problem is solved in the preferred embodiment of the present invention by acquiring cone beam projection data along a scan path comprised of two circular arcs disposed in perpendicular planes.

A second difficulty when producing a series of real-time images is the inability to acquire enough views in a specified time frame to satisfy the Nyquist criteria. This is called undersampling and the commonly believed consequence of undersampling within the prescribed scan path is streak artifacts in the reconstructed image. Most of the streak artifacts are static and are common to both the reference and contrast-enhanced images. We have discovered that undersampling by up to a factor of 50 is possible without producing clinically significant artifacts if a reference image is subtracted from the contrast enhanced image and if the images are isotropic 3D images which spread artifacts out in three dimensions rather than two. Streak artifacts common to both images are removed from the final image by subtracting the reference image from the contrast enhanced image. As a result, good 3D images can be produced with as few as 300 to 400 views of cone beam data.

A final difficulty with cone beam reconstruction methods is that the rays are divergent instead of parallel. The conventional projection-slice theorem establishes a bridge between the Fourier transform of parallel beam x-ray projections and a slice of the Fourier transform of an image object. In other words, a complete Fourier space depiction of the image object can be constructed from a superposition of the Fourier transform of the parallel beam projections. After the complete Fourier space of the image object is constructed, an inverse Fourier transform can be performed to reconstruct the image of the object. However, this is not valid for divergent rays produced by a cone beam. Various methods have been proposed to approximate the reconstructed image based on parallel beam principles. Disclosed here is a new cone beam reconstruction method which provides an exact reconstructed image from the cone beam data set.

The parallel beam projection-slice theorem tells us how each individual projection view contributes to the Fourier space depiction of an image object. Namely, Fourier space of the image object is constructed from the Fourier transform of the back-projection of the parallel beam rays in each projection view. In the parallel beam case, the image object is spatially shift-invariant. Therefore, it is natural to equally weigh the data during the back-projection. In other words, the detected x-ray attenuation data will be put back uniformly during the back-projection process to every point along the projection path. Thus, the Fourier transform of the back-projected data array only generates non-zero Fourier components in a plane perpendicular to the projections. Namely, a slice in Fourier space is generated by the Fourier transform of the projection data.

However, for the divergent beam projections, the equal weighting scheme is not appropriate because of the diverging nature of the beam. We have found that a proper weighting scheme is to multiply the measured data by a distance-dependent pre-weighting factor 1/r, where r is the distance from the x-ray source position to the back-projected point. After this pre-weighted back-projection step, the 2D projections become a fully 3D non-uniform data array within a cone. We take the Fourier transform of this weighted back-projection data array. A local Fourier space can be generated with the center of the Fourier space at the x-ray source location. In the cone beam case, this local Fourier transform is written as:

$$\begin{matrix}{G}_{3} [ \hat{k} , {y}^{->} ( t ) ] = {\int }_{ℜ} {\int }_{3} \int {d}^{3} r [ \frac{1}{r} \times g ( \hat{r} , {y}^{->} ( t ) ) ] {e}^{i 2 π {k}^{->} \cdot {r}^{->}} \\ = {\int }_{0}^{∞} d ll \tilde{f} ( l , \hat{k} ) {e}^{i 2 π l \hat{k} \cdot {y}^{->} ( t )}\end{matrix}$$

In the first line, the 1/r weighting on the acquired cone beam data g[\hat{r},\vec{y}(t)] has been highlighted in the square bracket. The vector \vec{y}(t) is used to label the x-ray tube position (focal spot). A hat is used to denote a unit vector and an arrow is used to denote a general vector. The second line of the above equation illustrates the relation between the Fourier transform of an image object \tilde{f}(l,\hat{k}) and the Fourier transform of the 1/r pre-weighted cone beam projections. We rebin the above partial Fourier transform data by introducing a new variable p:

p=\hat{k}·\vec{y}(t)

Then the above equation is transformed into:

$${G}_{3} ( p , \hat{k} ) = {\int }_{0}^{∞} d ll \tilde{f} ( l , \hat{k} ) {e}^{i 2 π lp}$$

For each of the projections, this procedure is repeated. For a specific Fourier space orientation \hat{k}, there may be more than one focal spot corresponding to the same p value. This represents the data redundancy in the divergent beam data acquisitions. Since each projection has generated an individual Fourier space around the x-ray source position, all local Fourier transforms are shifted to one fixed laboratory location. According to the shift theorem of the Fourier transform, this step requires an extra phase factor. After shifting, all the intermediate results are summed to obtain the desired Fourier transform of the target image object. Mathematically, this amounts to performing an inverse Laplace-Fourier transform to obtain the Fourier transform \tilde{f}(k, \hat{k}) from rebinned data G₃(p, \hat{k}):

$$\tilde{f} ( k , \hat{k} ) = \frac{1}{2 π {k}^{2}} \int d p cos ( 2 π kp ) \frac{d}{d p} Im {G}_{3} ( p , \hat{k} )$$

The integral is over all the possible rebinned p values. The symbol Im means the imaginary part.

The numerical implementation can be illustrated by the following pseudo code:

Step 1: for each acquired view t, calculate G₃(\hat{k},\vec{y}(t))

Step 2: rebin data to G₃(p,\hat{k}) by p=\hat{k}·\vec{y}(t)

Step 3: calculate \tilde{f}(k,\hat{k}) by using G₃(p,\hat{k}).

After these three steps, the physically measured cone beam projection data has been transformed into the Fourier space (i.e., k-space) version of the target image object. The 3D image of the object is then produced by Fourier transforming this k-space data.

There are alternative methods for reconstructing 3D images from acquired cone beam data sets. Two of these are described by:

Katsevich A. “A General Scheme For Constructing Inversion Algorithms For Cone Beam CT”, Int. J. Math and Math SCI. 2003; 21, 1305-1321; and

Chen G H. “An Alternative Derivation Of Katsevich's Cone-Beam Reconstruction Formula”, Med. Phys. 2003; 30.

These are generalized methods for use with cone beam data acquired with any scan path. Either of these generalized methods can be used by solving their general formula for the particular scan paths used herein.

Referring particularly to FIGS. 4-9, a number of different scan paths are employed by the above x-ray system to acquire sufficient cone-beam data. These scan paths are characterized by their ability to acquire sufficient cone-beam data from a large field of view (FOV) using the limited motions possible with the x-ray system.

Referring particularly to FIG. 4, the first scan trajectory is a circle segment 100 around the FOV combined with a linear segment 102 along one side of the FOV substantially perpendicular to the plane of the circle segment 100.

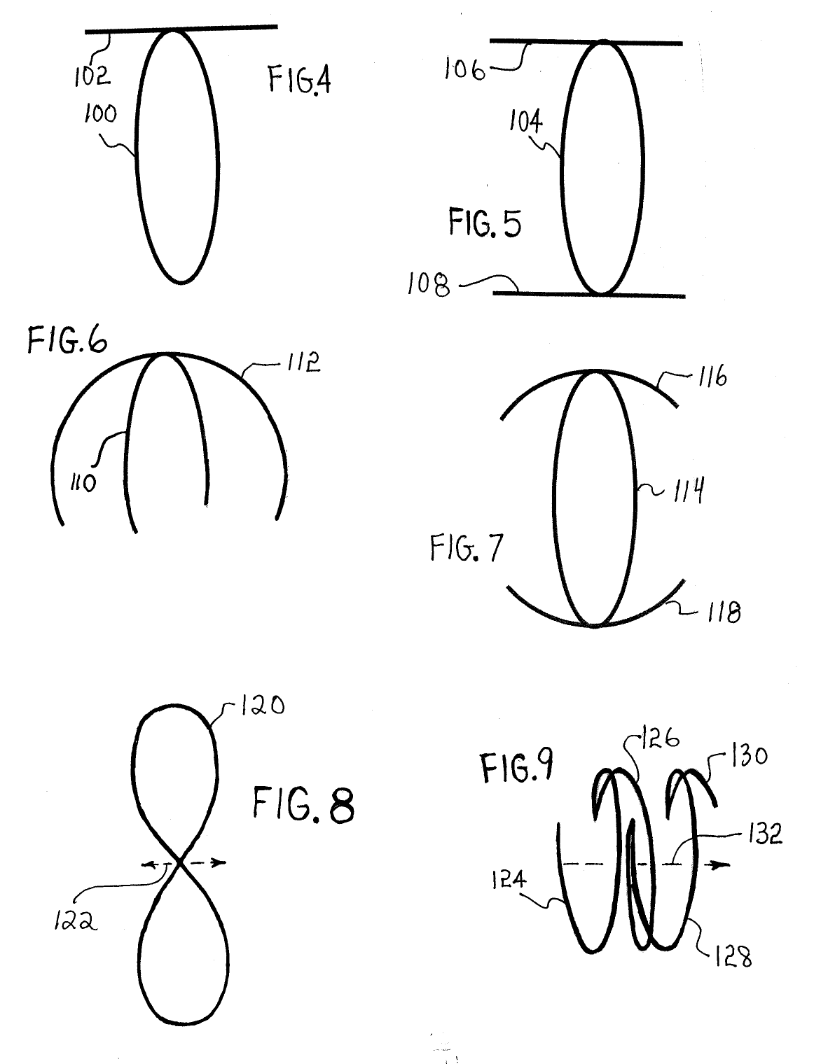

Referring particularly to FIG. 5, a second scan trajectory is comprised of a circular segment 104 combined with two linear segments 106 and 108. The linear segments 106 and 108 are substantially perpendicular to the plane of the circular segment 104 and they are disposed on opposite sides of the FOV.

The scanning trajectory shown in FIG. 4 is mechanically more convenient to implement than the scanning trajectory shown in FIG. 5. However, the trajectory shown in FIG. 5 provides more flexibility in using the cone-beam projection data in an image reconstruction procedure.

When the C-arm gantry does not allow for travel through an entire circle, the scanning trajectories shown in FIG. 4 and FIG. 5 become an arc and a straight line. In this case, the angular range of the arc portion needs to satisfy the short scan condition, i.e., 180° plus the cone angle.

Referring particularly to FIG. 1A, the circular segments 100 and 104 can be performed by rotating the C-arm 10 one revolution about its axis 26 and the linear segments 102, 106 and 108 can be performed by translating the table 16. With the scan path of FIG. 4 the table 16 is translated once while the C-arm 10 is stationary in one position. With the scan path of FIG. 5 the table 16 is translated once while the x-ray source is stationary on one side of the FOV and translated a second time while the x-ray source 12 is stationary on the opposite side of the FOV.

When the C-arm gantry cannot travel through an entire circle, the scan trajectory shown in FIG. 7 is reduced to the case shown in FIG. 6.

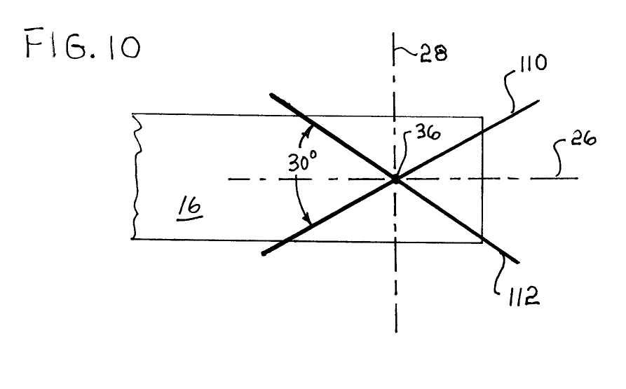

Referring particularly to FIGS. 1A, 6 and 10, another scan trajectory includes two circular arc segments 110 and 112 which lie in vertical planes that intersect at the system isocenter 36 and are oriented 15° to either side of the pivot axis 26. This orientation of the arcuate scan paths 110 and 112 enables them each to extend 180° plus the cone beam angle around the system isocenter 36 without engaging the patient table 16 or a subject positioned on the table 16. Sufficient data is thus acquired to reconstruct a 3D image without interfering with or moving the subject on the table 16.

A further scan path is shown in FIG. 7, where a circular segment 114 is performed and arc segments 116 and 118 are performed on opposite sides of the FOV. In this embodiment the arc segments 116 and 118 are performed by rocking the C-arm 10 while the x-ray source 12 is on one side of the FOV, and then rocking it again when the x-ray source 12 is rotated to the opposite side of the FOV.

Referring particularly to FIG. 8, yet another possible scan path that will acquire sufficient cone beam data from a large FOV is a warped circular segment 120. This path is produced by rotating the C-arm 10 one revolution around the axis 26, while at the same time translating the table 16 back and forth along the axis 26 as indicated by arrow 122. The degree of warp is determined by the amount of table translation, and this in turn is determined by the size of the FOV along the axis 26.

Referring particularly to FIGS. 1A and 9, another scan path that may be used when the FOV is extended along the axis 26 is comprised of a series of helical segments 124, 126, 128 and 130. This scan path is produced by rotating the C-arm 10 about axis 26 while the table 16 is translated in one direction indicated by arrow 132. The C-arm 10 is rotated one full revolution to produce helical segment 124 and then revolved in the opposite direction one full revolution to produce the helical segment 126. This pattern is repeated as many times as needed to cover the entire axial extent of the FOV.

The scanning trajectory shown in FIG. 9 is significantly different from other known helical trajectories. The difference lies in the fact that the connection between two helical segments 124 and 126 is twisted. Namely, after one helical segment 124 is traversed, the C-arm is revolved in the opposite direction while the patient table continues to translate in the same direction. In other helical scan trajectories there is no reversal of the rotation direction. therefore, the proposed new twisted helical trajectory is implementable on a C-arm gantry that does not have the commutation capability that enables multiple gantry rotations in a single direction.

1. An x-ray system which comprises:
a cone-beam x-ray source;
a two-dimensional x-ray detector array;
a drive mechanism for moving the x-ray source and x-ray detector in a programmed path about a subject positioned therebetween;
a computer for operating the x-ray source, x-ray detector and drive mechanism in accordance with a stored program for moving the x-ray source and x-ray detector along a scan path to acquire a data set from which a three-dimensional image of a field of view (FOV) is reconstructed, wherein the scan path includes:
a) a circular segment that extends around the FOV; and
b) a linear segment that extends in a direction substantially perpendicular to the plane of the circular segment.

2. The x-ray system of claim 1 in which the scan path includes:
c) a second linear segment located on the side opposite the FOV from the first linear segment.

3. An x-ray system which comprises:
a cone-beam x-ray source;
a two-dimensional x-ray detector array;
a drive mechanism for moving the x-ray source and x-ray detector in a programmed path about a subject positioned therebetween;
a computer for operating the x-ray source, x-ray detector and drive mechanism in accordance with a stored program for moving the x-ray source and x-ray detector along a scan path to acquire a data set from which a three-dimensional image is reconstructed, wherein the scan path includes:
a) a circular segment that extends around the FOV; and
b) an arc segment that extends in a direction substantially perpendicular to the plane of the circular segment.

4. The x-ray system of claim 3 in which the scan path includes:
c) a second arc segment located on the side opposite the FOV from the first arc segment.

5. An x-ray system which comprises:
a cone-beam x-ray source;
a two-dimensional x-ray detector array;
a drive mechanism for moving the x-ray source and x-ray detector in a programmed path about a subject positioned therebetween;
a computer for operating the x-ray source, x-ray detector and drive mechanism in accordance with a stored program for moving the x-ray source and x-ray detector along a scan path to acquire a data set from which a three-dimensional image of a field of view (FOV) is reconstructed, wherein the scan path includes a warped circular segment that extends around the FOV.

6. An x-ray system which comprises:
a cone-beam x-ray source;
a two-dimensional x-ray detector array;
a drive mechanism for moving the x-ray source and x-ray detector in a programmed path about a subject positioned on a table therebetween;
a computer for operating the x-ray source, x-ray detector and drive mechanism in accordance with a stored program for moving the x-ray source and x-ray detector along a scan path to acquire a data set from which a three-dimensional image of a field of view (FOV) is reconstructed, wherein the scan path includes a plurality of helical segments produced by rotating the x-ray source in alternating directions around the FOV.

7. The x-ray system as recited in claim 6 in which the helical segments are produced by also moving the table.

8. An x-ray system which comprises:
a cone-beam x-ray source;
a two-dimensional x-ray detector array;
a drive mechanism for moving the x-ray source and x-ray detector in a programmed path about a subject positioned between the x-ray source and the x-ray detector array and disposed on a table that is aligned along a pivot axis;
a computer for operating the x-ray source, x-ray detector array and drive mechanism in accordance with a stored program for moving the x-ray source and x-ray detector array along a scan path to acquire a data set from which a three-dimensional image of a field of view (FOV) is reconstructed, wherein the scan path includes two arcuate scan path segments that lie in intersecting planes which are disposed at opposite angles with respect to the pivot axis such that neither the x-ray source or x-ray detector array engage the table or the subject as they are moved along the two arcuate scan path segments.

9. The x-ray system as recited in claim 8 in which each arcuate scan path is a circular path that extends around its center 180° plus the cone-beam angle.

10. The x-ray system as recited in claim 8 in which the intersecting planes are vertical and are disposed at substantially equal, but opposite angles with respect to the pivot axis.

11. The x-ray system as recited in claim 10 in which the angle between the intersecting planes is substantially less than 90°.

12. The x-ray system as recited in claim 11 in which the angle between the intersecting planes is substantially 30°.
